(19) United States
(12) Patent Application Publication
(10) Pub. No.: US 2012/0172973 A1
(43) Pub. Date: Jul. 5, 2012

(54) SELF-EXPANDING OCCLUSION DEVICE

(75) Inventors: Michael Deckard, Solsberry, IN (US); James Taylor, Bloomington, IN (US); Mark Magnuson, Bloomington, IN (US); James Ryan Randolph, Bloomington, IN (US)

(73) Assignee: Cook Medical Technologies LLC, Bloomington, IN (US)

(21) Appl. No.: 13/338,725

(22) Filed: Dec. 28, 2011

Related U.S. Application Data
(60) Provisional application No. 61/428,708, filed on Dec. 30, 2010.

(51) Int. Cl. A61F 2/82; A61M 29/00

(52) U.S. Cl. 623/1.16; 606/192

(57) ABSTRACT

An occlusion device for implantation within a body lumen includes a distal end, a proximal end, and an expandable member extending from the distal end to the proximal end. The expandable tubular member defines a passageway extending from the distal end to the proximal end. The expandable tubular member includes a plurality of struts that are joined to adjacent struts to form a plurality of closed shapes each defining a closed path. The plurality of struts are joined to form a plurality of fixed joints so that adjacent struts are pivotable around respective one of the fixed joints when an external force is applied to the expandable tubular member. The tubular member defines an ellipsoid shape.

### CROSS-REFERENCE TO RELATED APPLICATIONS

This application claims the benefit of U.S. Provisional Application No. 61/428,708, filed on Dec. 30, 2011, entitled “SELF-EXPANDING OCCLUSION DEVICE,” the entire contents of which are incorporated herein by reference.

### FIELD

The present invention relates to medical devices. More particularly, the invention relates to an occlusion device for occluding a lumen of a blood vessel.

### BACKGROUND

Vascular occlusion devices are surgical implants that are placed within the vascular system of a patient. There are a number of reasons why it may be desirable to occlude a vessel. For example, the site of a stroke or other vascular accident can be treated by placing an occlusion device proximal of the site to block the flow of blood to the site, thereby alleviating leakage at the site. An aneurysm can be treated by the introduction of an occlusion device through the neck of the aneurysm. Tumours can be treated by occluding the flow of blood to a target site of interest.

Several known occlusion devices include a coil having fibers, threads or strands attached to the coil. Such occlusion devices act to block the flow of blood through a vessel by the formation of an embolus in the vessel. While these occlusion devices can provide effective occlusion, they suffer from the disadvantage that blood flow continues until the embolus has been formed, thus requiring additional time before effective occlusion is obtained.

Plug-style occlusion devices have also been developed. While these devices are intended to provide a physical barrier to blood flow, and thereby stop blood flow more quickly, known devices are generally bulky and often require thrombosis to obtain reliable occlusion.

### SUMMARY

The present invention provides an improved occlusion for use in various medical procedures.

In one form, an occlusion device for implantation within a body lumen includes a distal end, a proximal end, and an expandable tubular member extending from the distal end to the proximal end and defining a passageway extending from the distal end to the proximal end. The expandable tubular member is a one-piece member and includes a plurality of struts. Adjacent struts are joined to form a fixed joint so that the adjacent struts are pivotable around the fixed joint and remain joined at the fixed joint when an external force is applied to the expandable tubular member. The expandable tubular member includes a distal portion including the distal end, a proximal portion including the proximal end, and an intermediate portion disposed between the distal portion and the proximal portion. The intermediate portion has a diameter greater than that of the proximal portion and the distal portion in the expanded state such that the proximal portion and the distal portion are tapered proximally and distally, respectively, from the intermediate portion to define an ellipsoid shape in the expanded state.

In another form, an occlusion device for implantation within a body lumen includes a distal end, a proximal end, and a plurality of tubular members disposed between the distal end and the proximal end. The plurality of tubular members each define an ellipsoid shape. The utmost proximal tubular member includes the proximal end. The utmost distal tubular member includes the distal end. A plurality of bridge members are disposed between the plurality of tubular members and have a diameter smaller than that of the tubular members. The tubular members and the bridge members are alternately arranged and connected. A passageway extends from the proximal end to the distal end and inside the tubular members and the bridge members. The plurality of tubular members each include a plurality of struts and adjacent struts thereof are joined to form a fixed joint. The adjacent struts are pivotable around the fixed joints and remain joined at the fixed joints when an external force is applied to the tubular members.

In still another form, a method of manufacturing a vascular occlusion device in accordance with the teachings of the present disclosure includes: providing a tubular member; cutting the tubular member to form a plurality of cuts, the plurality of cuts defining a plurality of struts, adjacent struts thereof being joined to form a fixed joint; expanding the tubular member to a predetermined shape including a proximal portion, a distal portion and an intermediate portion between the proximal portion and the distal portion, wherein the proximal portion and the distal portion are tapered proximally and distally, respectively, from the intermediate portion such that the tubular member defines an ellipsoid shape; thermally setting the tubular member at a transition temperature so that the tubular member automatically returns to the predetermined shape when the tubular member is not restrained.

Further objects, features, and advantages of the present invention will become apparent from consideration of the following description and the appended claims when taken in connection with the accompanying drawings.

### DRAWINGS

FIG. 1 is a side view of an occlusion device in accordance with the teachings of the present disclosure, wherein the occlusion device is in a collapsed state;

FIG. 2 is a side view of an occlusion device in accordance with the teachings of the present disclosure, wherein the occlusion device is in an expanded state;

FIG. 3 is a side view of an alternate form of an occlusion device in accordance with the teachings of the present disclosure;

FIG. 4 is a side view of the occlusion device of FIG. 3, which is just released in the blood vessel and is about to expand;

FIG. 5 is a side view of another alternate form of an occlusion device in accordance with the teachings of the present disclosure;

FIG. 6 is a side view of an occlusion device in accordance with yet another embodiment of the present disclosure; and

FIG. 7 is an enlarged view of portion A of FIG. 6;

FIG. 8 is a side view of a delivery and retrieval assembly for use with the occlusion device, in accordance with the teachings of the present disclosure;

FIG. 9 is an exploded view of the delivery and retrieval assembly of FIG. 8, in accordance with the teachings of the present disclosure;

FIG. 10 is a side view of an occlusion device and a delivery and retrieval assembly, showing retrieval of the occlusion device; and

FIG. 11 is a flowchart of a method of manufacturing an occlusion device in accordance with the teaching of the present disclosure.

### DETAILED DESCRIPTION

The following provides a detailed description of currently preferred embodiments in accordance with the teachings of the present disclosure. The description is not intended to limit the invention in any manner, but rather serves to enable those skilled in the art to make and use the invention. The present disclosure generally provides an occlusion device and an occlusion device delivery system that may be used by a physician to deliver an occlusion device into the vasculature of a patient.

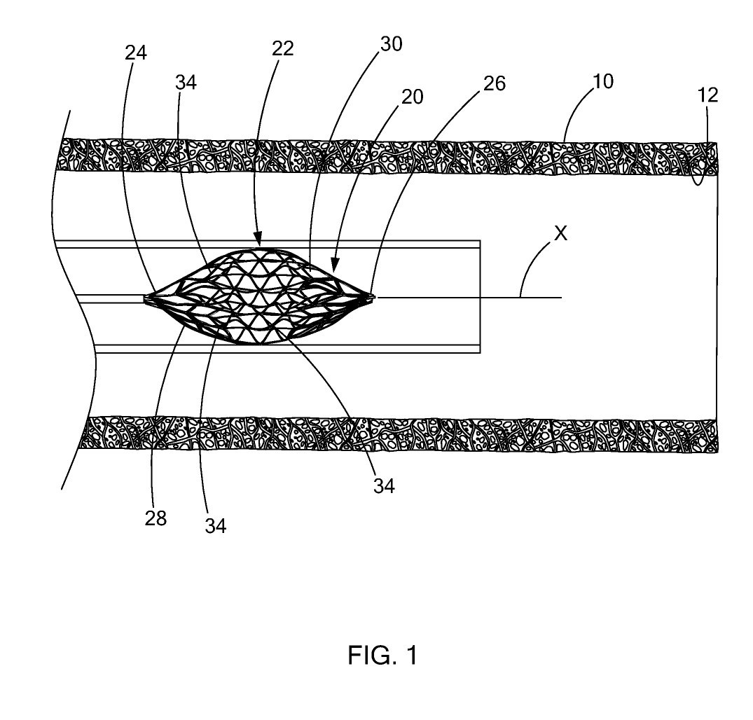

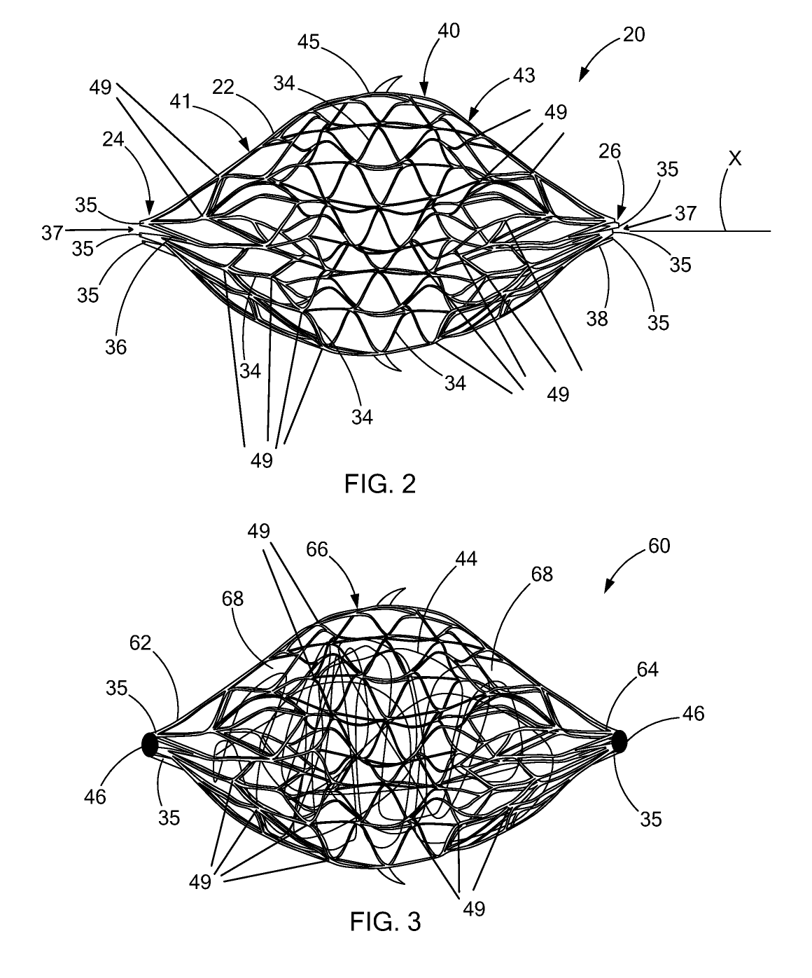

Referring to FIGS. 1 and 2, an occlusion device 20 in accordance with the teachings of the present disclosure includes a tubular member 22 having a proximal end 24 and a distal end 26. The occlusion device 20 is movable between a radially collapsed state (FIG. 1) and a radially expanded state (FIG. 2) and is used to occlude vessels or be placed in aneurisms.

The tubular member 22 may be a braided nitinol Z-Stent/cannula and may include a tubular wall 28 defining an interior passageway 30 extending from the proximal end 24 to the distal end 26 along a longitudinal axis X. A plurality of cuts are formed through the tubular wall 28 to define a plurality of radially expandable struts 34. The plurality of struts 34 are biased to expand.

As clearly shown in FIG. 2, the tubular member 22 has a substantially ellipsoid configuration when in the deployed/expanded state and includes a proximal tapered portion 36 including the proximal end 24, a distal tapered portion 38 including the distal end 26, and an intermediate portion 40 therebetween. The proximal and distal tapered portions 36 and 38 each define a cone or tapered shape and are tapered proximally and distally, respectively, from the intermediate portion 40. The intermediate portion 40 has an outside diameter greater than the maximum diameter of the proximal and distal tapered portions 36 and 38. The proximal end 24 and the distal end 26 may be closed or substantially closed so that the proximal end 24 and the distal end 26 each define a pointed end or tip. In the embodiment shown in FIG. 2, the proximal end 24 and the distal end 26 are “substantially closed” because the adjacent struts at the proximal end 24 and the distal end 26 are arranged closely to define a pointed end having a small opening 37.

The plurality of cuts are formed through the tubular wall 28 to form a plurality of struts 34 defining a strut pattern. The strut pattern defines a plurality of closed shapes. A “closed shape” as used herein means a shape defined by a closed path including a starting point and an end portion that coincides with the starting point. For example, a circle, a triangle, a polygon, or a ring is a closed shape.

In the present embodiment, the plurality of struts 34 are configured so that each strut 34 is joined to an adjacent strut. When a strut is joined to an adjacent strut, a proximal end or a distal end of the strut is joined to a proximal end or a distal end of the adjacent strut to form a fixed joint 49. Adjacent struts 34 are joined at a fixed joint 49 and are not loosely attached. Therefore, the adjacent struts 34 remain joined at the fixed joint 49 and are pivotable around the fixed joint 49 when the tubular member 22 is movable between a collapsed state and an expanded state or when an external force (e.g., from the blood flow) is applied to the tubular member 22.

The plurality of struts 34 of the tubular member 22 define a plurality of closed shapes and a plurality of fixed joints 49 that form corners of the closed shapes. The plurality of struts 34 and the fixed joints 49 are integrally formed from a single-piece and integral tubular body.

In one example, the strut pattern formed by the cuts includes a plurality of closed shapes, which are polygonal shapes, wherein the fixed joints 49 form corners of the polygonal shapes. As used herein, the term “polygonal shape” refers to a shape defined by a closed path composed of a finite sequence of line segments. While polygonal shapes are illustrated in this embodiment, it is understood that any shape can be formed as long as each strut is joined to another strut to define a closed shape, i.e., a shape defined by a closed path. In the present embodiment, the plurality of polygonal shapes are joined to adjacent polygonal shapes along both a circumferential direction and a longitudinal direction of the tubular member 22 at the fixed joints 43. Therefore, the opposing ends of each strut are joined to one end of an adjacent strut to define a closed shape except for the ends of the struts 34 at the proximal end 24 and the distal end 26. In this embodiment, the cuts formed through the tubular wall 28 may extend the entire length of the tubular member 22 to define a plurality of strut ends 35 (FIG. 2) at each of the proximal and distal ends 24, 26.

It is understood that the plurality of polygonal shapes may be joined by fixed joints only along the circumferential direction to define a plurality of rings; the rings, each defining a plurality of polygonal shapes, may be connected loosely along the longitudinal direction without departing from the scope of the present disclosure.

The intermediate portion 40 includes a first tapered portion 41 connected to the proximal tapered portion 36 and a second tapered portion 43 connected to the distal tapered portion 38. The joining part 45 of the first tapered portion 41 and the second tapered portion 43 defines the maximum diameter of the intermediate portion 40. The intermediate portion 40 can be expanded to have an outside diameter of approximately 20 mm and can be collapsed to have an outside diameter of approximately 1.35 mm (4 French catheter). The maximum outside diameter of the intermediate portion 40 in the expanded state is preferably greater than or substantially equal to that of the blood vessel 10.

The occlusion device 20 may include a plurality of barbs 50 extending radially and outwardly from the occlusion device 20 at around the largest diameter of the intermediate portion 40, i.e., the joining part 45 of the first tapered portion 41 and the second tapered portion 43. The barbs 50 aid in attachment of the occlusion device 20 to the vessel wall 12 of the blood vessel 10. The barbs 50 may be particularly useful if the occlusion device 20 is not dimensioned larger than the diameter of the blood vessel 10.

The occlusion device 20 of the present disclosure, when deployed in the body vessel, has sufficient radial force to stop migration. The struts 34 are joined to adjacent ones of the struts 34 to form a plurality of closed shapes defined by the fixed joints 49. The closed shapes are joined along both the longitudinal direction and the circumferential direction and remain joined at the fixed joints 49. The struts 34 that are joined at the fixed portions 49 are not separated when an external force is applied. When an external force is applied to the tubular member, the fixed joints 49 allow the force to be transmitted from one strut to another strut through the fixed joints 49 to more evenly distribute the force among the struts 34. Therefore, the occlusion device 20 of the present disclosure imparts higher radial force on the interior wall of the blood vessel than that of a prior art occlusion device formed by woven wires. In the prior art occlusion device formed by woven wires, the wires are woven to intersect one another and are in loose contact with adjacent wires. The interesting points are not joined and thus are incapable of transmitting and distributing force when a force is applied to the occlusion device. Therefore, the prior art occlusion device imparts less radial force on the interior wall of the blood vessel to resist migration.

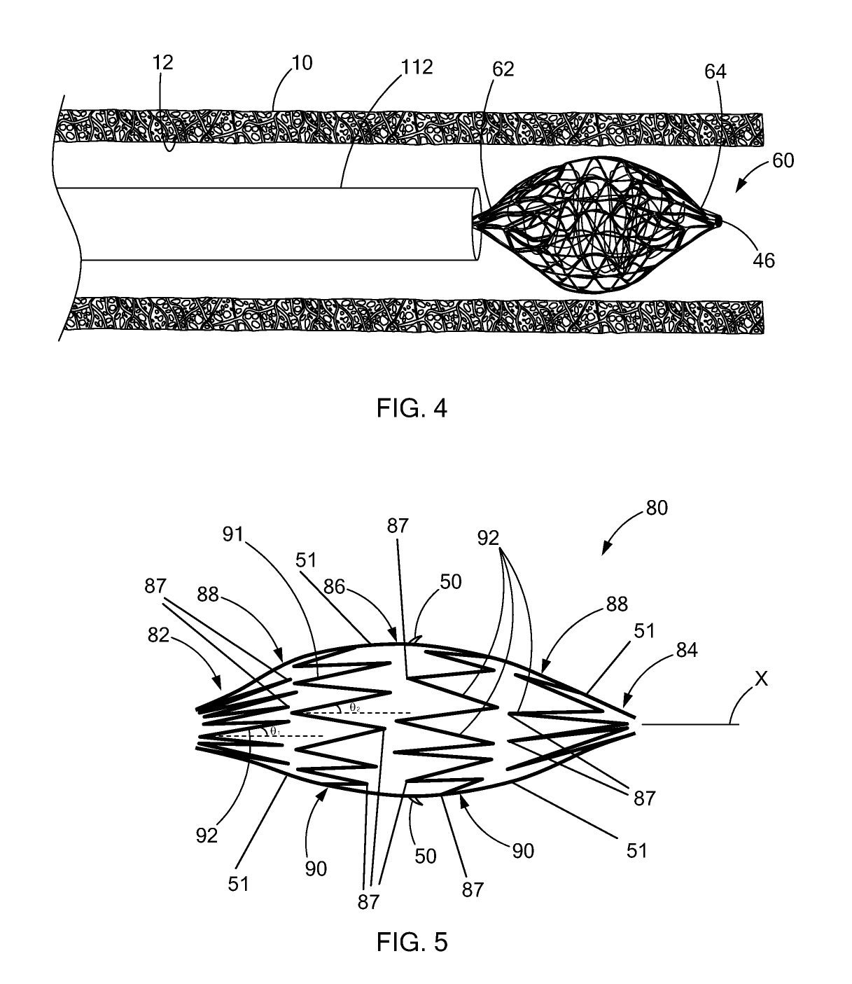

Moreover, the occlusion device 20 is actually one laser-cut stent/braided stent/cannula, rather than a device including a plurality of woven or welded wires. Therefore, the occlusion device 20 of the present disclosure has higher mechanical strength than that of a prior art occlusion device due to the integral and one-piece structure of the tubular member 22. With the proximal tapered portion 36, the occlusion device 20 can be retracted into a sheath of a catheter at least when the occlusion device 20 is not completely deployed if the desired location is not the correct location (see FIG. 4).

Referring to FIGS. 3 and 4, an alternate form of the occlusion device 60 is similar to the occlusion device 20 of FIGS. 1 and 2 except for the provision of an occluding material 44 and the weld joints 46. More specifically, the occlusion device 60 includes a proximal taped portion 62, a distal tapered portion 64, and an intermediate portion 66 therebetween. The occlusion device 60 further includes an occluding material 44 disposed in the occlusion device 60. The occluding material 44 may include, for example, Dacron® fibers disposed within the interior passageway 30 to aid in occlusion. The occluding material 44 may be formed from other suitable materials known or contemplated by one of ordinary skill in the art, including but not limited to Thorolon®, Gore-tex®, PET, PTFE, silk, nylon, shish, SIS and the like.

The occluding material 44 may further include sheets of SIS material disposed within the interior passageway 68. Alternatively, SIS sheet material may be disposed along the inner or outer surface of the occlusion device 60 by any suitable means known in the art, including but not limited to, bonding with silicon adhesive. The SIS sheet may have any length depending on the applications and may preferably have a length between 5 mm and 100 mm.

As shown in FIG. 3, the strut ends 35 adjacent to the proximal end 62 and the distal end 64 are arranged closely to form a pointed end or tip and are further pinched closed to form closed ends. In other words, the struts ends 35 are joined to form a closed end. For example, the strut ends 35 may be welded together to form a spherical weld joint 46 at each of the proximal end 62 and the distal end 64 such that the proximal and distal ends 62 and 64 do not define a small opening 37 as in the embodiment of FIG. 2. The ellipsoid shape of the occlusion device 60, together with the spherical weld joint 46 at the proximal end 62 thereof, aid in delivery and retrieval of the occlusion device 60. This is because the cone shape formed by the proximal tapered portion adjacent to the proximal end 62 and/or the proximal spherical weld joint 46 are more easily grasped by a grasping delivery member during delivery and/or retrieval of the occlusion device 20. Moreover, as clearly shown in FIG. 4, with the proximal tapered portion, the occlusion device 60 can be retracted into a sheath of a catheter at least when the occlusion device 60 is not completely deployed if the desired location is not the correct location.

Referring to FIG. 5, an alternate form of the occlusion device 80 is similar to that of FIG. 2 except for the pattern of the struts. Similar to the occlusion devices 20 and 60 of FIGS. 2 and 3, the occlusion device 80 defines a substantially ellipsoid shape and is formed by laser-cutting a tubular member to form a plurality of struts. The plurality of struts are arranged and joined to define a plurality of closed shapes, which are a plurality of zigzag rings. The zig-zag rings each define a closed path along the circumference of the tubular member of the occlusion device 80.

More specifically, the occlusion device 80 includes a proximal tapered portion 82, a distal tapered portion 84 and an intermediate portion 76 therebetween. The proximal tapered portion 82 and the distal tapered portion 84 each define a first zig-zag ring 88 having a first diameter. The intermediate portion 86 defines two second zig-zag rings 90 having a second diameter. The first diameter is smaller than the second diameter.

In the first zig-zag rings 88 at the proximal tapered portion 82 and the distal tapered portion 84, the struts 92 are oriented to extend substantially along the longitudinal axis X but define a first angle θ₁ relative to the longitudinal axis X. In the second zig-zag rings 90 at the intermediate portion 86, the struts 92 are oriented to extend substantially along the longitudinal axis X but define a second angle θ₂ relative to the longitudinal axis X. The second angle θ₂ is greater than the first angle θ₁. Therefore, the struts 92 in the first zig-zag rings 88 are more closely disposed adjacent to one another than the struts 92 in the second zig-zag rings 90.

The struts 92 in the first zig-zag rings 88 are joined to adjacent ones of the struts 92 along the circumferential direction of the occlusion device 80 to form a plurality of fixed joints 87, thereby forming the first zig-zag rings 88. The struts 92 in the second rings 90 are joined to adjacent ones of the struts 92 along the circumferential direction to form a plurality of fixed joints 87, thereby forming the second zig-zag rings 90. The first and second zig-zag rings 88 and 90 have closed configuration/shape and are connected in series along the longitudinal axis X at a few points by a plurality of longitudinal struts 51 to increase expansion flexibility of the occlusion device 80. Similarly, the first and second zig-zag rings 88 and 90 are connected to the plurality of longitudinal struts 51 to form a plurality of fixed joints 87. The struts 51 are pivotable around respective ones of the fixed joints 87 when an external force is applied or when the occlusion device 80 is movable between the collapsed state and the expanded state or in

In the present embodiment, the zig-zag rings 88 and 90, which are closed shapes extending around the circumferential direction of the occlusion device 80, allow the occlusion device 80 to provide a radial force greater than that of a prior art occlusion device 80 formed by woven wires. When an external force is applied to the zig-zag rings 88 and 90, the fixed joints 87 of the zag-zag rings 88 and 90 help evenly distribute the force among the struts 92 within the same zag-zag rings 88 and 90. Moreover, the external force applied to one zig-zag ring 88 or 90 may be transmitted to an adjacent zag-zag ring 90 or 88 through the longitudinal struts 51 due to the provision of the fixed joints 87. Therefore, the entire structure of the occlusion device imparts sufficient radial force on the interior wall of the blood vessel in response to an external force to resist migration of the occlusion device 80 in the blood vessel.

It is understood that the one zig-zag ring may be connected loosely to an adjacent zig-zag ring without departing from the scope of the present disclosure as long as a plurality of fixed joints are defined in the same zig-zag ring to help transmit and distribute an external force to resist migration.

Similar to the occlusion device of FIG. 3, an occluding material 44 may be placed inside the first and second zig-zag rings 88 and 90 to aid in occlusion. In addition, barbs 50 may be formed on the intermediate portion 86 to increase engagement between the occlusion device 80 and the vessel walls 12 of the blood vessel 10.

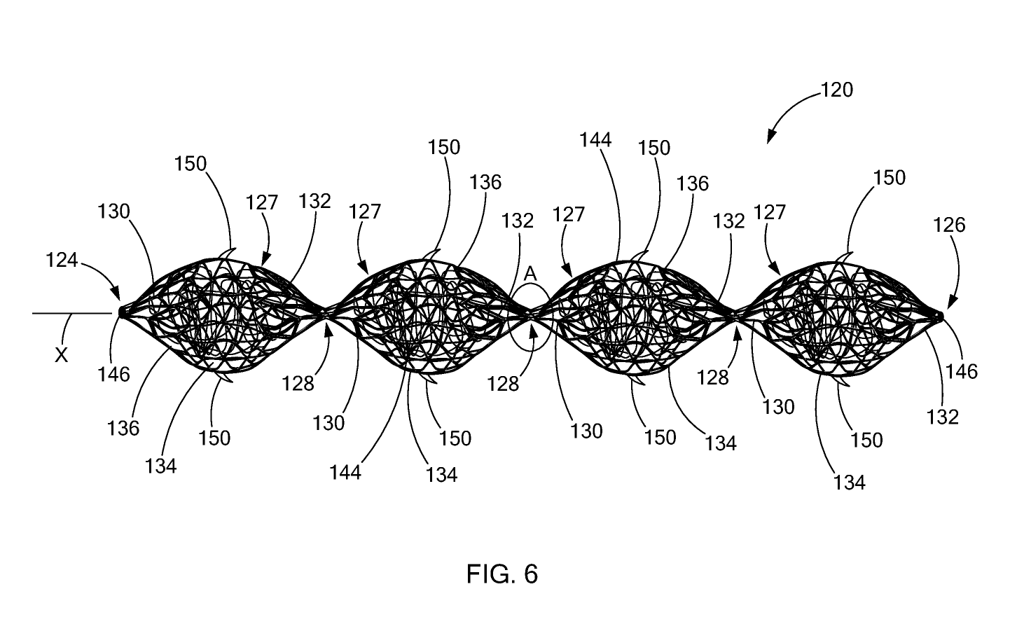

Referring to FIG. 6, an alternate form of an occlusion device 120 defines a proximal end 124 and a distal end 126. The occlusion device 120 includes a plurality of expandable tubular members 127 and a plurality of bridge members 128 which are joined in series along the longitudinal axis X of the occlusion device 120. The utmost proximal expandable tubular member 127 includes the proximal end 124. The utmost distal expandable tubular member 127 includes the distal end 126. One of the expandable tubular members 127 is joined to an adjacent one of the expandable tubular members 127 by one of the bridge members 128. Therefore, the expandable tubular members 127 and the bridge members 128 are alternatively arranged and connected along the longitudinal axis X of the occlusion device 120. The bridge members 128 may be expandable or non-expandable. The plurality of expandable tubular members 127 each include a proximal tapered portion 130, a distal tapered portion 132 and an intermediate portion 134 therebetween. The intermediate portions 134 of the plurality of expandable tubular members 127 commonly define a maximum diameter of the occlusion device 120 in the expanded state. The bridge members 128 may each have a diameter sufficiently smaller than the diameter of the intermediate portions 134 of the tubular members 127 so that the bridge members 128 form pointed end or tip of the adjacent ones of the tubular members 127. The plurality of tubular members 127 and the bridge members 128 jointly define a passageway extending along the longitudinal axis X of the occlusion device 120 and between the proximal end 124 and the distal end 126 of the occlusion device 120.

The occlusion device 120 with a series of expandable tubular members 126 in this embodiment is suitable for use in legs to cut off blood flow in a larger blood vessel or in multiple sections. The occlusion device 120 with a single expandable tubular member (FIG. 2, 3 or 5) may be used in a smaller vessel.

The occlusion device 120 includes a plurality of struts 136 extending substantially along the longitudinal axis X of the occlusion device 120. The struts 136 are biased to expand to an expanded state. The occlusion device 120 may include an occluding material 144 in the expandable tubular members 127 to aid in occlusion.

The proximal end 124 and the distal ends 126 of the occlusion device 120 may be open like the occlusion device 20 of FIG. 2, or terminated with a spherical weld joint 146, like the occlusion device 60 of FIG. 3. In this embodiment, the strut ends at respective proximal and distal ends 124, 126 are welded together, forming a spherical weld joint 146 at each end. The conical shape of the proximal portion 130 of the proximal most expandable member 127 and the spherical weld joint 146 at the proximal end 124 aid in delivery and retrieval of the occlusion device 120. This is because the cone shape of the proximal tapered portion 136 of the proximal most expandable member 127 and/or the spherical weld joint 124 at the proximal end 124 are more easily grasped by a grasping delivery member during delivery and/or retrieval of the occlusion device 120.

Barbs 150 may be provided at the intermediate portions 134 of the expandable tubular members 127 to improve engagement between the occlusion device 120 and the vessel walls 12 of the blood vessels 10.

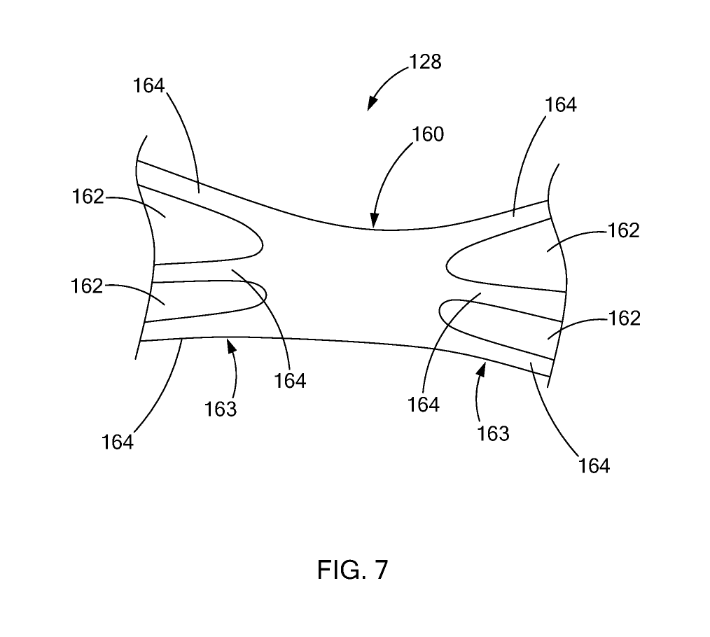

Referring to FIG. 7, the bridge member 128 of the occlusion device 120 is shown to include a tubular portion 160. The tubular portion 160 is laser-cut to form a plurality of longitudinal slits 162 at opposing ends 163, thereby defining a plurality of longitudinal struts 162. The longitudinal struts 164 are joined to the struts 136 of an adjacent proximal tapered portion 130 or an adjacent distal tapered portion 132. The bridge member 128 may be expandable or non-expandable when the occlusion device 120 is in the expanded state.

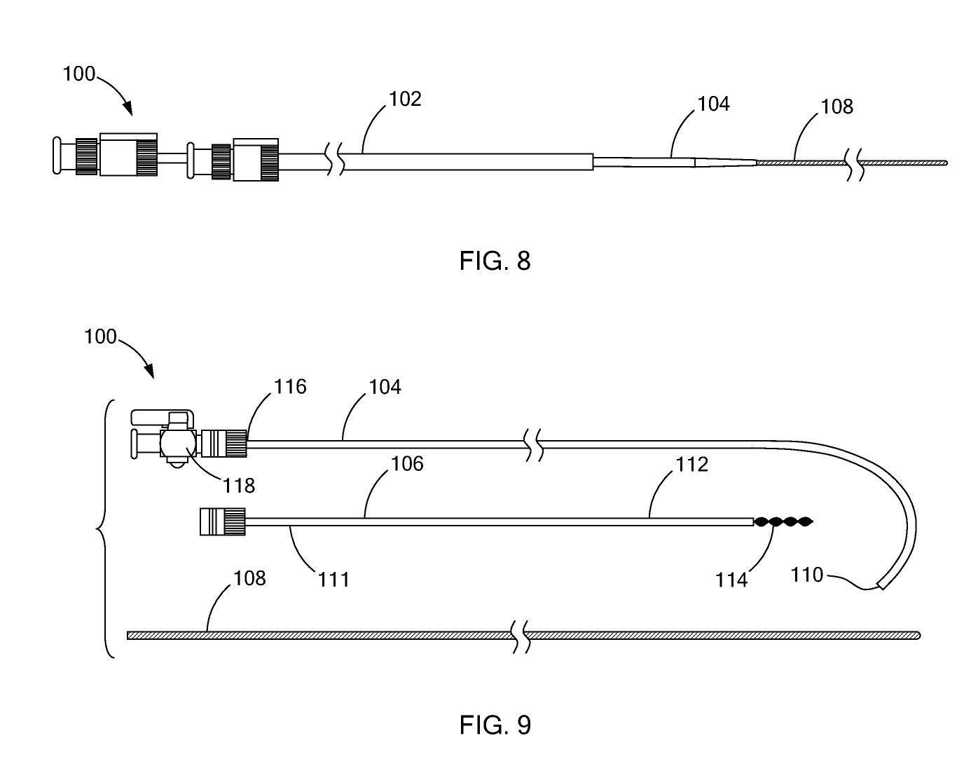

Referring to FIGS. 8 and 9, a delivery assembly 100 for introducing and retrieving the occlusion device for occluding a body vessel in accordance with another embodiment of the present invention. As shown, the delivery assembly 100 includes a polytetrafluoroethylene (PTFE) introducer sheath 102 for percutaneously introducing an outer sheath 104 into a body vessel. Of course, any other suitable material for the introducer sheath 102 may be used without falling beyond the scope or spirit of the present invention. The introducer sheath 102 may have any suitable size, for example, between about three-french to eight-french. The introducer sheath 102 serves to allow the outer sheath 104 and an inner member or catheter 106 to be percutaneously inserted to a desired location in the body vessel. The inner member may also include, for example, a stylet. The introducer sheath 102 receives the outer sheath 104 and provides stability to the outer sheath 104 at a desired location of the body vessel. For example, the introducer sheath 102 is held stationary within a common visceral artery, and adds stability to the outer sheath 104, as the outer sheath 104 is advanced through the introducer sheath 102 to an occlusion area in the vasculature. The outer sheath 104 has a body extending from a proximal end 116 to a distal end 110, the body being tubular and including a sheath lumen extending therethrough.

As shown, the assembly 100 may also include a wire guide 108 configured to be percutaneously inserted within the vasculature to guide the outer sheath 104 to the occlusion area. The wire guide 108 provides the outer sheath 104 with a path to follow as it is advanced within the body vessel. The size of the wire guide 108 is based on the inside diameter of the outer sheath 104 and the diameter of the target body vessel.

When the distal end 110 of the outer sheath 104 is at the desired location in the body vessel, the wire guide 108 is removed and the occlusion device 114, having a proximal segment contacting a distal portion 112 of the inner catheter 106, is inserted into the outer sheath 104. The inner catheter 106 is advanced through the outer sheath 104 for deployment of the occlusion device 114 (similar to any of the occlusion devices 20, 60, 80 and 120 of FIGS. 1-7) through the distal end 110 to occlude the body vessel during treatment of, for example, an aneurism, or to otherwise occlude a body vessel. The catheter 1806 extends from a proximal portion 111 to a distal portion 112 and is configured for axial movement relative to the outer sheath 104. In this example, the distal portion 112 is shown adjacent to an occlusion device 114 (similar to any of the occlusion devices described above). Thus, before deployment, the occlusion device 114 is coaxially disposed within the lumen of the outer sheath 104 and removably coupled to the distal portion 112 of the catheter 106, or in the alternative, the occlusion device 114 is merely pushed by, but not coupled to, the distal portion 112 of the catheter 106.

The outer sheath 104 further has a proximal end 116 and a hub 118 to receive the inner catheter 106 and occlusion device 114 to be advanced therethrough. The size of the outer sheath 104 is based on the size of the body vessel in which it percutaneously inserts, and the size of the occlusion device 114.

In this embodiment, the occlusion device 114 and inner catheter 106 are coaxially advanced through the outer sheath 104, following removal of the wire guide 108, in order to position the occlusion device 114 to occlude the body vessel. The occlusion device 114 is guided through the outer sheath 104 by the inner catheter 106, preferably from the hub 118, and exits from the distal end 110 of the outer sheath 104 at a location within the vasculature where occlusion is desired. Thus, the occlusion device 114 is deployable through the distal end 110 of the outer sheath 104 by means of axial relative movement of the catheter 106. In order to more easily deploy the occlusion device 114 into the body vessel, the occlusion device 114 may have a slippery coating, such as Silicone or slipcoating.

Likewise, this embodiment may also retrieve the occlusion device 114 by positioning the distal end 110 of the outer sheath 104 adjacent the deployed device 114 in the vasculature. The inner catheter 106 is advanced through the outer sheath 104 until the distal portion 112 protrudes from the distal end 110 of the outer sheath 104. The distal portion 112 is coupled to a proximal end of the occlusion device 114, after which the inner catheter 106 is retracted proximally, drawing the occlusion device 114 into the outer sheath 104.

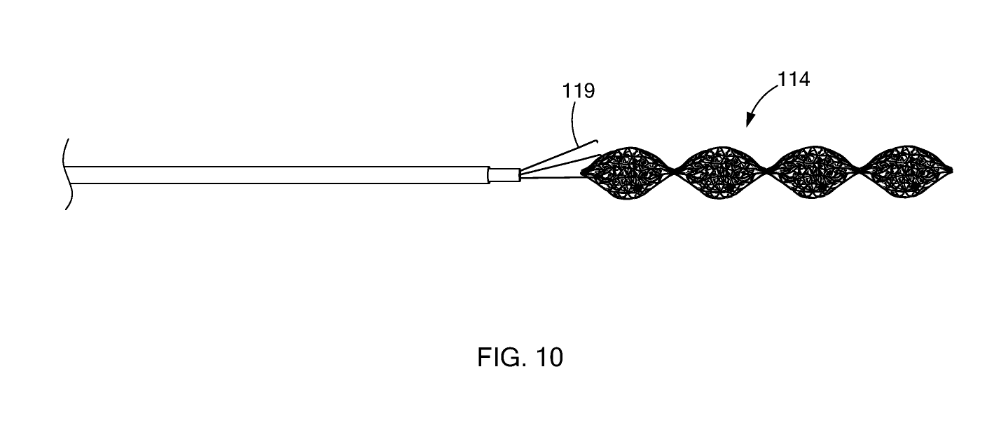

Referring to FIG. 10, to retrieve the occlusion device 114 from the blood vessel, a clip device 119 may be used with the delivery assembly 100 for retrieving the occlusion device 114. With the cone-shape proximal end of the occlusion device 114, the clip device 119 can grab the proximal end of the occlusion device 114 and retrieve the occlusion device 114 into the sheath.

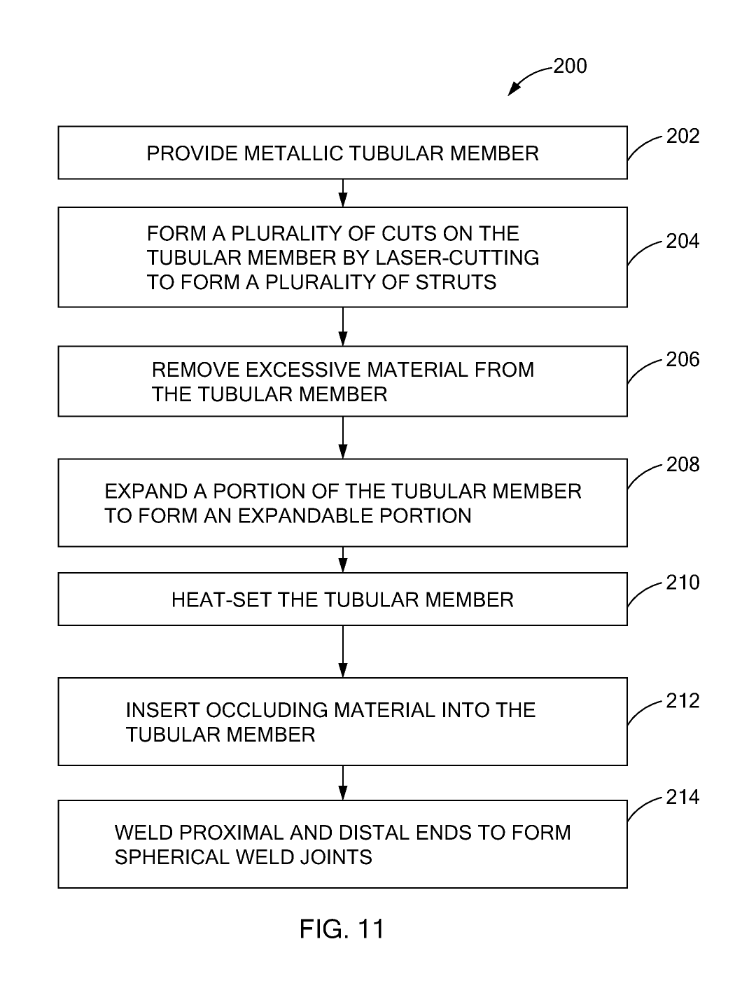

Referring to FIG. 11, a method 200 of manufacturing an occlusion device is described. A small-diameter tubular member is provided in step 202. The tubular member may be Nitinol braid/Stent/cannula. The tubular member is made of a material that allows the occlusion device to be self-expanding. For example, the tubular member may be formed from a shape-memory alloy (such as nitinol), a shape-memory polymer, or may be formed from other self-expandable materials, such as spring steel. The tubular member may be a preformed braid. The occlusion device with a preformed braid facilitates blocking off blood flow through the vascular system may be placed in any location that needs to be blocked off or placed in an aneurism.

Next, laser-cutting is performed on the tubular member to cut a discontinuous pattern of cuts in the tubular member in step 204. Therefore, the discontinuous pattern of cuts define a plurality of struts that are joined to form a plurality of closed shapes. Excessive material is removed from the interior and exterior surfaces of the tubular member in step 206. In one embodiment, the tubular member becomes a laser cut nitinol cannula.

The tubular member is further processed to be collapsible in an undeployed state and expandable in a deployed state. The tubular member is expanded to an ellipsoid shape in step 208. Portions along the tubular member are expanded to provide one or more expandable tubular sections in step 208.

To set the occlusion device, metal dies and metal clamps are arranged in an alternate manner so that the expandable tubular members/sections of the occlusion device are formed at where the metal dies are located and that the bridge members are formed at where the metal clamps are located. The metal clamps restrain the expansion of the bridge members when the expandable tubular sections are expanded. For an occlusion device having only one expandable tubular member (FIG. 2), the clamps are placed around the distal end and the proximal end. For occlusion device having a plurality of expandable tubular members/sections (FIG. 6), the metal clamps are placed around the proximal end, the distal end and the bridge members.

The metallic tubular member is then placed in ionized bath and thermally set to the expanded state at a transition temperature in step 210. Thermally-treating the tubular member allows the tubular member to remember the shape of the tubular member in the expanded state after the tubular member is collapsed. Therefore, when the tubular member is pushed down, it will automatically come back to the ellipsoid shape (FIG. 2) or multiple ellipsoid shapes (FIG. 6). The metal dies are later removed.

The occlusion material, such as Dacron fibers, may be inserted into the occlusion device in step 212. For example, fibers may be placed within the occlusion device by, for example, inserting a straw-like tubular member through the small openings 37 of the proximal end 24 and the distal end 26. Optionally, the proximal and distal ends may be closed off by welding the strut ends 35 of the struts adjacent to the proximal and distal ends to create spherical weld joints in step 214 to complete the occlusion device.

The manufacturing method of the present disclosure has a better control of the shape of the occlusion device. For example, a desired shape of the occlusion device in the expanded state may be achieved by properly changing the shape of the dies and the locations of the dies and the clamps. Moreover, the diameter of the occlusion device in the collapsed state can be made as small as possible (for example, 4F catheter (1.35 mm)) by choosing a metallic tubular member having the desired small diameter. The small diameter of the tubular member in the collapsed state is not limited by the number of wires formed on the tubular member.

Further, the occlusion device manufactured by the manufacturing method of the present disclosure has higher strength than that of a prior art occlusion device due to the more unitary and integral structure. While the drawings show that the struts look like wires, these struts are not wires. These struts are laser-cut cannula—cannula pre-cut to a certain shape and heat-set, as opposed to a prior art occlusion device that uses weaving wires. The use of Dacron fibers inside or outside the tubular member or sewed SIS onto inside or outside helps with growing into walls. The occlusion device can be used to occlude blood vessels or any part that needs occlusion, if doctor wants to shut off certain area.

As a person skilled in the art will readily appreciate, the above description is meant as an illustration of the implementation of the principles of this invention. This description is not intended to limit the scope or application of this invention in that the invention is susceptible to modification variation and change, without departing from the spirit of this invention, as defined in the following claims.

1. An occlusion device for implantation within a body lumen comprising:
a distal end;
a proximal end; and
an expandable tubular member extending from the distal end to the proximal end and defining a passageway extending from the distal end to the proximal end, the expandable tubular member being a one-piece member and including a plurality of struts, wherein adjacent struts thereof are joined to form a fixed joint so that the adjacent struts are pivotable around the fixed joint and remain joined at the fixed joint when an external force is applied to the expandable tubular member, the expandable tubular member including
a distal portion including the distal end,
a proximal portion including the proximal end, and
an intermediate portion disposed between the distal portion and the proximal portion, wherein the intermediate portion has a diameter greater than that of the proximal portion and the distal portion in the expanded state such that the proximal portion and the distal portion are tapered proximally and distally, respectively, from the intermediate portion to define an ellipsoid shape in the expanded state.

2. The occlusion device of claim 1, wherein the plurality of struts define a plurality of fixed joints and a plurality of closed shapes each defining a closed path, the fixed joints forming corners of the closed shapes.

3. The occlusion device of claim 2, wherein the closed shapes include polygons, the polygons being joined to adjacent polygons at least along a circumferential direction of the occlusion device.

4. The occlusion device of claim 3, wherein the polygons are joined to adjacent polygons along both the circumferential direction and a longitudinal direction of the occlusion device.

5. The occlusion device of claim 2, wherein the closed shapes include a plurality of zig-zag rings formed along a circumferential direction of the occlusion device.

6. The occlusion device of claim 5, wherein the plurality of zig-zag rings are connected by a plurality of longitudinal struts extending along a longitudinal direction of the occlusion device.

7. The occlusion device of claim 1, wherein the struts at each of the proximal end and the distal end are joined to form a pointed end.

8. The occlusion device of claim 7, wherein the pointed end is a weld joint.

9. The occlusion device of claim 1, further comprising an occluding material inside the tubular member.

10. The occlusion device of claim 9, wherein the occluding material includes one of strands of SIS material, sheets of SIS material, and Dacron fibers.

11. The occlusion device of claim 1, wherein the expandable tubular member is a stent.

12. The occlusion device of claim 1, further comprising barbs on the intermediate portion.

13. The occlusion device of claim 1, further comprising a plurality of expandable tubular members and a plurality of bridge members between the proximal end and the distal end, the bridge members having a diameter smaller than that of the expandable tubular members, the expandable tubular members and the bridge members being alternately arranged and connected along a longitudinal direction of the occlusion device.

14. The occlusion device of claim 13, wherein the bridge members each define a pointed end.

15. The occlusion device of claim 13, wherein the plurality of expandable tubular members and the plurality of bridge members jointly define the passageway extending along the longitudinal direction of the occlusion device.

16. The occlusion device of claim 13, wherein the plurality of expandable tubular members each define an ellipsoid shape.

17. The occlusion device of claim 13, wherein the plurality of expandable tubular members commonly define a maximum diameter of the occlusion device in the expanded state.

18. The occlusion device of claim 17, wherein the maximum diameter is approximately 20 mm when the occlusion device is in the expanded state.

19. An occlusion device for implantation within a body lumen, comprising:
a distal end;
a proximal end;
a plurality of tubular members disposed between the distal end and the proximal end and each defining an ellipsoid shape, the utmost proximal tubular member including the proximal end, the utmost distal tubular member including the distal end;
a plurality of bridge members disposed between the plurality of tubular members and having a diameter smaller than that of the tubular members, the tubular members and the bridge members being alternately arranged and connected; and
a passageway extending from the proximal end to the distal end and inside the tubular members and the bridge members,
wherein the plurality of tubular members each include a plurality of struts and adjacent struts thereof are joined to form a fixed joint, the adjacent struts pivotable around the fixed joints and remaining joined at the fixed joints when an external force is applied to the tubular members.

20. The occlusion device of claim 19, wherein the struts include a plurality of fixed joints and a plurality of closed shapes, the fixed joints forming corners of the closed shapes.

21. The occlusion device of claim 19, wherein the struts define a plurality of zig-zag rings and a plurality of longitudinal struts, the plurality of zig-zag rings being oriented circumferentially around a longitudinal direction of the occlusion device and connected by the plurality of longitudinal struts, the longitudinal struts extending along the longitudinal direction of the occlusion device.

22. A method of manufacturing an occlusion device, comprising:
providing a tubular member;
cutting the tubular member to form a plurality of cuts, the plurality of cuts defining a plurality of struts, adjacent struts thereof being joined to form a fixed joint;
expanding the tubular member to a predetermined shape including a proximal portion, a distal portion and an intermediate portion between the proximal portion and the distal portion, wherein the proximal portion and the distal portion are tapered proximally and distally, respectively, from the intermediate portion such that the tubular member defines an ellipsoid shape; and
thermally setting the tubular member at a transition temperature so that the tubular member automatically returns to the predetermined shape when the tubular member is not restrained.

23. The method of claim 22, wherein the predetermined shape includes a plurality of ellipsoid shapes connected along a longitudinal direction of the occlusion device.

24. The method of claim 22, wherein the plurality of struts define a plurality of fixed joints and a plurality of polygonal shapes, the fixed joints forming corners of the polygonal shapes.

25. The method of claim 22, wherein the struts define a plurality of zig-zag rings and a plurality of longitudinal sturts, the zig-zag ring oriented circumferentially around a longitudinal direction of the occlusion device and connected by the plurality of longitudinal struts along the longitudinal direction.

26. The method of claim 22, wherein the tubular member includes a proximal end and a distal end, further comprising welding the struts at the proximal end and the distal end so that proximal ends of the struts at the proximal end of the tubular member and distal ends of the struts at the distal end of the tubular member are welded to form a weld point.

27. The method of claim 22, further comprising expanding the tubular member and heat-setting the tubular member to form a plurality of expandable tubular members and a plurality of bridge members each having a diameter smaller than that of the tubular members, wherein the expandable tubular members and the bridge members are alternately arranged and connected.
